(19) United States
(12) Patent Application Publication
(10) Pub. No.: US 2022/0176287 A1
(43) Pub. Date: Jun. 9, 2022

(54) WALL-MOUNTED AIR PURIFIER

(71) Applicant: SHENZHEN ANTOP TECHNOLOGY CO., LTD, Shenzhen (CN)

(72) Inventors: Ruidian YANG, Shenzhen (CN); Xianquan LIAO, Shenzhen (CN)

(73) Assignee: SHENZHEN ANTOP TECHNOLOGY CO., LTD, Shenzhen (CN)

(21) Appl. No.: 17/171,264

(22) Filed: Feb. 9, 2021

(30) Foreign Application Priority Data
Dec. 9, 2020 (CN) 202022935470.0

(51) Int. Cl. B01D 46/00; B01D 46/12; B01D 46/16; B01D 46/44; A61L 9/20

(52) U.S. Cl. CPC B01D 46/0005; B01D 46/12; B01D 46/0023; B01D 46/0049; B01D 46/0028; B01D 46/16; A61L 2209/12; A61L 9/20; B01D 2267/40; B01D 2273/30; B01D 2279/65; A61L 2209/14; B01D 46/44

(57) ABSTRACT

The present application is to provide a wall-mounted air purifier, including a housing, a filter installed in the housing, and a fan installed in the housing. The housing is provided with an air inlet and an air outlet, the air outlet is arranged at a position corresponding to an outlet of the fan, the filter is arranged between the air inlet and the fan, and the air inlet is arranged at a position corresponding to a rear side of a bottom of the housing, and the air outlet is arranged at a position corresponding to a front side of the bottom of the housing.

### CROSS-REFERENCE TO RELATED APPLICATIONS

The present application claims priority to Chinese Patent Applications No. 202022935470.0 filed on Dec. 9, 2020, the entire content of which is incorporated herein by reference in its entirety.

### FIELD

The present application relates to the field of air purification technology, more particularly, relates to a wall-mounted air purifier.

### BACKGROUND

As people pay more attention to the environment, more and more air purifiers are used. The current air purifiers are divided into floor-standing and wall-mounted. The wall-mounted air purifier is generally installed on the wall and will not occupy the floor space of the room, and is more and more favored by users. However, the current wall-mounted air purifier generally uses top air intake, which requires a large range of circulating air purification in the room, and dust is easy to fall into the housing.

### SUMMARY

The purpose of the embodiment of the present application is to provide a wall-mounted air purifier to solve the problem that the wall-mounted air purifier requires a large-scale circulating purification of room air, and dust is easy to fall into the housing in the related technology.

In order to achieve the above objective, the technical solution adopted by the embodiment of the present application is to provide a wall-mounted air purifier, including a housing, a filter installed in the housing, and a fan installed in the housing, the housing is provided with an air inlet and an air outlet, the air outlet is arranged at a position corresponding to the outlet of the fan, the filter is arranged between the air inlet and the fan, and the air inlet is arranged at a position corresponding to the rear side of the bottom of the housing, and the air outlet is arranged at a position corresponding to the front side of the bottom of the housing.

In an optional embodiment, the bottom of the housing has an inclined surface extending backward and upward, and the air inlet is arranged on the inclined surface.

In an optional embodiment, the bottom of the housing is provided with a baffle protruding downward at both ends of the inclined surface respectively.

In an optional embodiment, the wall-mounted air purifier further includes a filter screen covering the air inlet, and the filter screen is detachably installed on the housing.

In an optional embodiment, the wall-mounted air purifier further includes a grid plate for supporting the filter screen, and the grid plate is detachably installed on the housing.

In an optional embodiment, the bottom of the housing has an air outlet surface extending forward and upward, and the air outlet is arranged on the air outlet surface.

In an optional embodiment, the air outlet surface is arcuate, or the air outlet surface is inclined.

In an optional embodiment, an ultraviolet light source is further installed in the housing, and the ultraviolet light source is arranged between the air inlet and the fan.

In an optional embodiment, a control panel is arranged on the front side of the housing, and the control panel is inclined from top to bottom toward the rear side of the housing.

In an optional embodiment, the fan includes a wind cover installed in the housing, a through-flow wind wheel installed in the wind cover, and a motor that drives the wind wheel to rotate.

Beneficial effects of the wall-mounted air purifier provided by the embodiment of the present application are summarized as follows: compared with the prior art, the wall-mounted air purifier filter in the present application purifies the air by arranging a filter in the housing; an air inlet is arranged on the rear side of the bottom of the housing to prevent dust from falling into the housing; and an air outlet is arranged on the front side of the bottom of the housing. In use, air can be guided and gathered through the wall to enter the air inlet, so that the air under the housing can circulate upwards into the housing to improve the air purification capacity, and can blow the air in the area below the housing to better flow and better purify the air, and it can also make the users under the wall-mounted air purifier breathe purified air more quickly, thus enhancing user experience.

Further areas of applicability will become apparent from the description provided herein. The description and specific examples in this summary are intended for purposes of illustration only and are not intended to limit the scope of the present disclosure.

### DRAWINGS

In order to more clearly describe the technical solutions in the embodiment of the present application, the following will briefly introduce the accompanying drawings used in the embodiments or exemplary technical descriptions. Obviously, the accompanying drawings in the following description are merely some of the embodiments of the present application, for those of ordinary skill in the art, other drawings can be obtained based on these drawings without creative work.

FIG. 1 is a schematic diagram of the wall-mounted air purifier provided by the embodiment of the present application;

FIG. 2 is a schematic diagram of the filter and filter screen in the wall-mounted air purifier of FIG. 1 when being taken out;

FIG. 3 is a schematic diagram of the bottom view of the wall-mounted air purifier of FIG. 1;

FIG. 4 is a perspective sectional view of the structure along the line A-A in FIG. 3;

FIG. 5 is a schematic sectional view of the wall-mounted air purifier in FIG. 4;

FIG. 6 is a schematic diagram of the wall-mounted air purifier in FIG. 1 when the hanging plate is separated;

FIG. 7 is a schematic diagram 1 of an exploded structure of the wall-mounted air purifier of FIG. 1;

FIG. 8 is a schematic diagram 2 of an exploded structure of the wall-mounted air purifier of FIG. 1;

FIG. 9 is a schematic sectional view of the wall-mounted air purifier provided by the embodiment 2 of the present application;

FIG. 10 is a schematic sectional view of the wall-mounted air purifier provided by the embodiment 3 of the present application;

FIG. 11 is a schematic diagram of the light shield in the wall-mounted air purifier provided by the embodiment 4 of the present application;

FIG. 12 is a schematic sectional view of the wall-mounted air purifier provided by the embodiment 5 of the present application;

FIG. 13 is a schematic sectional view of the wall-mounted air purifier provided by the embodiment 6 of the present application;

FIG. 14 is a schematic sectional view of the wall-mounted air purifier provided by the embodiment 7 of the present application;

FIG. 15 is a schematic diagram of an enlarged structure of the transmitting head in FIG. 14;

FIG. 16 is a schematic sectional view of the wall-mounted air purifier provided by the embodiment 8 of the present application; and

FIG. 17 is a schematic diagram of the wall-mounted air purifier provided by the embodiment 9 of the present application.

The main reference numbers of the drawings in the FIGS. are as follows:

100—wall-mounted air purifier;

10—housing; 11—bottom housing; 111—inclined surface; 112—air inlet; 113—baffle; 114—air outlet surface; 115—air outlet; 116—socket; 117—sliding plate; 12—casing; 121—hidden trunking; 122—hanging buckle; 123—opening window; 124—cover plate;

21—filter; 211—magnetic block; 212—magnetic member; 22—partition plate; 221—chute; 23—filter screen; 24—grid plate;

31—fan; 311—wind cover; 312—through-flow wind wheel; 313—motor;

41—ultraviolet light source; 411—ultraviolet tube; 412—lamp holder; 413—ultraviolet LED module; 42—light shield; 421—vent; 422—first opening; 423—blocking sheet; 424—second opening; 425—third opening;

51—hanging board; 511—hook; 52—control panel; 53—negative ion generator; 530—transmitting head; 531—negative ion release brush; 532—support; and 533—guide plate.

Corresponding reference numerals indicate corresponding parts throughout the several views of the drawings.

### DETAILED DESCRIPTION

Example embodiments will now be described more fully with reference to the accompanying drawings.

In order to make the technical problems to be solved, technical solutions, and beneficial effects by the present application clearer, the present application will be further described in detail below with reference to the accompanying drawings and embodiments. It should be understood that the specific embodiments described here are only used to explain the present application, and are not used to limit the present application.

It should be noted that when an element is referred to as being “fixed to” or “arranged on” another element, it can be directly on the other element or indirectly on the other element. When an element is said to be “connected to” another element, it can be directly connected to the other element or indirectly connected to the other element.

In addition, the terms of “first” and “second” are only used for descriptive purposes, and cannot be understood as indicating or implying relative importance or implicitly indicating the number of indicated technical features. Thus, the features defined with “first” and “second” may explicitly or implicitly include one or more of these features. In the description of the present application, “a plurality of” means two or more, unless otherwise specifically defined. The meaning of “several” is one or more than one, unless otherwise specifically defined.

In the description of the present application, it should be understood that the orientation or positional relationship indicated by the terms of “center”, “length”, “width”, “thickness”, “upper”, “lower”, “front”, “rear”, “left”, “right”, “vertical”, “horizontal”, “top”, “bottom”, “inner”, “outer” etc. is based on the orientation or positional relationship shown in the drawings, and is convenient to describe the present application and simplify the description, instead of indicating or implying that the device or element referred to must have a specific orientation, be constructed and operated in a specific orientation, and therefore cannot be understood as a restriction on the present application.

In the description of the present application, it should be noted that the terms of “installation”, “connection” and “connecting” should be understood in a broad sense, unless otherwise clearly specified and limited. For example, it can be a fixed connection or a detachable connection, or integral connection; it can be mechanical connection or electrical connection; it can be directly connected or indirectly connected through an intermediate medium, it can be the internal communication of two components or the interaction relationship between two components. For those of ordinary skill in the art, the specific meaning of the above-mentioned terms in the present application can be understood according to specific circumstances.

The reference to “one embodiment”, “some embodiments” or “embodiments” described in the present application specification means that one or more embodiments of the present application include specific features and structures or characteristics described in combination with the embodiment. Therefore, the words that “in one embodiment”, “in some embodiments”, “in some other embodiments”, “in some other embodiments”, etc. appearing in different places in this specification are not necessarily all referring to the same embodiment, but mean “one or more but not all embodiments”, unless otherwise specifically emphasized. In addition, in one or more embodiments, specific features, structures, or characteristics may be combined in any suitable manner.

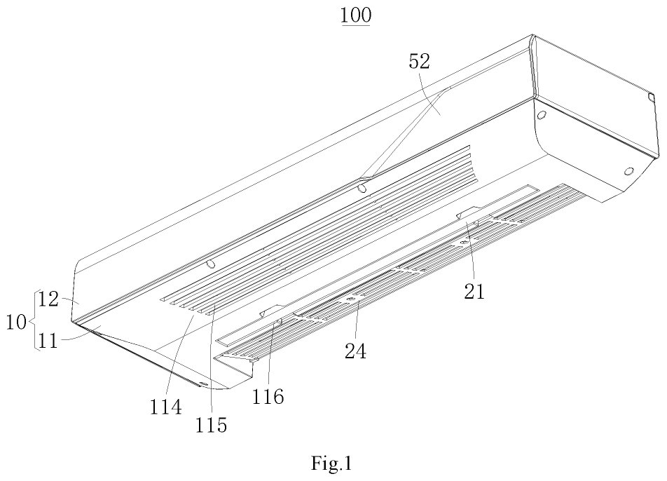

For the convenience of description, referring to FIG. 1 and FIG. 5. The definition in the present application: when housing 10 is installed on a wall, one side that housing 10 is close to the wall is the rear side of the housing 10 and the wall-mounted air purifier 100, and one side that is away from the wall is the front side of housing 10 and the wall-mounted air purifier 100.

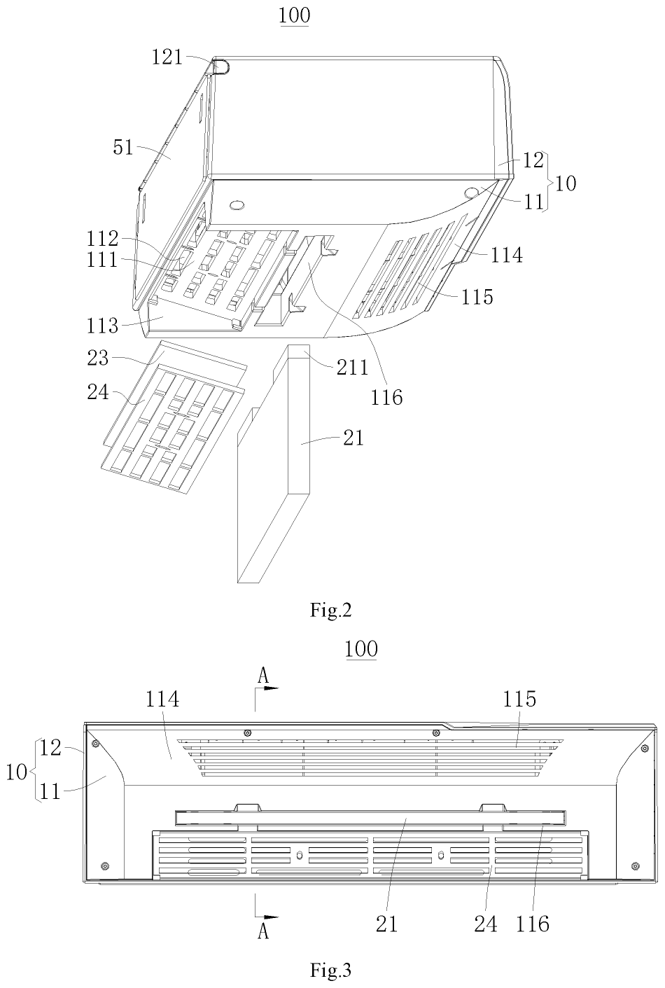

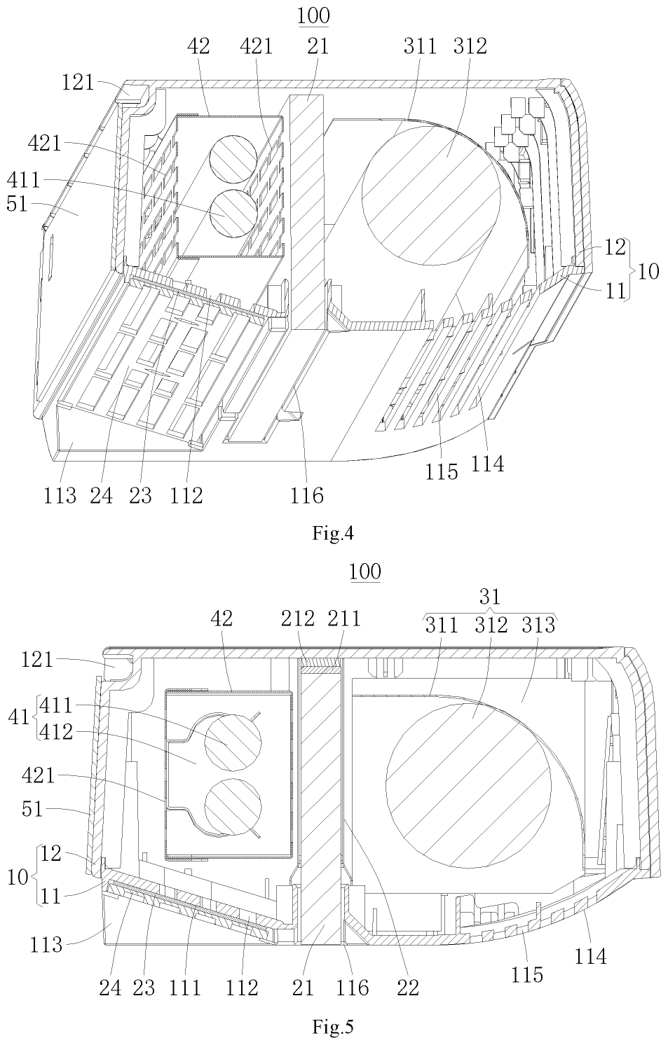

Referring to FIGS. 1 to 5, where FIG. 1 is a perspective schematic diagram of the wall-mounted air purifier 100 of this embodiment. FIG. 2 is a schematic diagram of the filter 21 and the filter screen 23 of the wall-mounted air purifier 100 of this embodiment when they are separated from the housing 10. FIG. 3 is a schematic bottom view of the wall-mounted air purifier 100 of this embodiment. FIG. 4 is a schematic sectional view of the structure taken along A-A in FIG. 3, and FIG. 4 is a perspective sectional view of the wall-mounted air purifier 100. FIG. 5 is a schematic sectional view of the structure taken along A-A in FIG. 3.

Referring to FIG. 4 and FIG. 5, the wall-mounted air purifier 100 provided by the present application will now be described. The wall-mounted air purifier 100 includes a housing 10, a filter 21 and a fan 31; the filter 21 and the fan 31 are installed in housing 10, and the filter 21 and the fan 31 are supported and protected by housing 10. The housing 10 is provided with an air inlet 112 and an air outlet 115. The air outlet 115 is arranged at the position corresponding to the outlet of fan 31. The filter 21 is arranged between the air inlet 112 and fan 31. When fan 31 works, air can enter the housing 10 from air inlet 112, and then pass through the filter and is purified by filter 21 and is blown out by fan 31 from air outlet 115 to purify the air. In addition, the filter 21 is arranged between the air inlet 112 and the fan 31, and the air can be filtered and purified by the filter 21 before entering the fan 31, thereby reducing the adhesion of impurities onto the fan 31.

Referring to FIG. 1 and FIG. 5, the air inlet 112 on the housing 10 is arranged at a position corresponding to the rear side of the bottom of the housing 10. Since the wall-mounted air purifier 100 is generally installed on the wall, the air inlet 112 is arranged at the bottom of the housing 10, so that the air under the wall-mounted air purifier 100 can be directly sucked and purified, so as to more quickly purify the air in the user's active area to provide the purified air to users faster and better. The arrangement that the air inlet 112 is arranged at a position corresponding to the rear side of the bottom of the housing 10, the wall can guide and gather the air entering the air inlet 112, so that the air below the housing 10 circulates upwards into the housing 10 to improve the air purification capacity. And it can better prevent impurities from falling into the air inlet 112.

The air outlet 115 of the housing 10 is arranged at a position corresponding to the front side of the bottom of housing 10, so that the air in the area under housing 10 can flow better, so as to better purify the air, and it can also make users under the wall-mounted air purifier 100 can breathe the purified air faster, improving user experience.

Compared with the prior art, the wall-mounted air purifier 100 provided by the present application can filtering purify the air by installing a filter 21 in the housing 10; installing an air inlet 112 on the rear side of the bottom of the housing 10 can avoid dust falling into the housing 10; and an air outlet 115 is arranged on the front side of the bottom of housing 10. In use, air can be guided and collected through the wall to enter air inlet 112, so that the air under housing 10 can circulate upwards into housing 10 and improve the air purification capacity. And it can also blow the air in the area under housing 10 to better flow, so as to better purify the air, and it can also make users under the wall-mounted air purifier 100 breathe the purified air faster, improving user experience.

In one embodiment, referring to FIGS. 4 and 5, the wall-mounted air purifier 100 further includes an ultraviolet light source 41; the ultraviolet light source 41 is installed in the housing 10, and the ultraviolet light source 41 is supported and protected by the housing 10, and the ultraviolet light source 41 is arranged between the air inlet 112 and the fan 31, the air is sterilized by ultraviolet light source 41 before entering fan 31 to improve the air purification function and promote cleaner air.

In one embodiment, referring to FIGS. 2 and 4, the bottom of housing 10 has an inclined surface 111, the air inlet 112 is arranged on the inclined surface 111, and the inclined surface 111 extends backward and upward from the bottom of housing 10. The inclined surface 111 is arranged and the air inlet 112 is arranged on the inclined surface 111. When the housing 10 is installed on the wall, the inclined surface 111 and the wall can form a trumpet-like structure, that is, a trough structure with an open bottom and a contractive upper side. The air can be collected better, so that the air can enter the air inlet 112 more quickly, so that the air below the housing 10 can circulate upwards into the housing 10 to improve the air purification capacity.

In one embodiment, the inclined surface 111 has a planar shape to facilitate processing. It is understandable that the inclined surface 111 may also be arranged to be arcuate or curved.

In one embodiment, the bottom of housing 10 is provided with a baffle 113 protruding downwards at both ends of the inclined surface 111 respectively, so that it can better cooperate with the wall to form a cavity with a reduced area from bottom to top to better gather air and facilitate air to enter the air inlet 112 faster, so that the air below housing 10 circulates upwards into housing 10 to improve air purification capacity.

In an embodiment, referring to FIGS. 4 and 5, the filter 21 may be a primary filter, a high-efficiency filter, an activated carbon filter, and so on.

In an embodiment, referring to FIGS. 2, 4 and 5, the wall-mounted air purifier 100 further includes a filter screen 23, which is detachably installed on the housing 10, and the filter screen 23 covers the air inlet 112 to filter air through the filter screen 23, and to reduce dust, hair and other impurities from entering the housing 10. The filter screen 23 can be detachably installed on the housing 10 for disassembly, cleaning or replacement.

In one embodiment, the wall-mounted air purifier 100 further includes a grid plate 24, which is detachably installed on the housing 10, and the grid plate 24 is used to support the filter screen 23. The grid plate 24 is arranged to support the filter screen 23, and further to facilitate the installation of the filter screen 23 on the housing 10.

In one embodiment, the grid plate 24 can be fixed on the housing 10 by screws to facilitate disassembly. The grid plate 24 can also be snapped onto the housing 10.

In one embodiment, the filter screen 23 and the grid plate 24 can be made as one piece, that is, the filter screen 23 and the grid plate 24 are an integral structure, so that the filter screen 23 is supported by the grid plate 24 so as to install the filter screen 23 on the housing 10. In some embodiments, the filter screen 23 can be arranged separately, and the filter screen 23 can be directly fixed on the housing 10. For example, the four sides or a plurality of positions all around of the filter screen 23 can be fixed on the housing 10 by screws, magnetic attraction, buckles, etc. This structure can reduce the cost, and it is convenient to remove the filter screen 23 and clean it.

In one embodiment, the bottom of housing 10 has an air outlet surface 114, the air outlet 115 is arranged on the air outlet surface 114, and the air outlet surface 114 extends forward and upward from the bottom of the housing 10, so that the air blown by the air outlet 115 is blown toward down and front side the bottom of the wall-mounted air purifier 100 which can better drive the air flow under the wall-mounted air purifier 100, and the purified air can directly cover a larger area, so that the user can breathe the purified air faster and improve the user experience.

In one embodiment, the air outlet surface 114 is arcuate to reduce the edges and corners on the front side of the bottom of the housing 10 and to improve safety and aesthetics, and also enable the air blown by the air outlet 115 to cover a larger area. The air outlet surface 114 can also be inclined to facilitate processing.

In one embodiment, the housing 10 includes a bottom housing 11 and a casing 12 covering the bottom housing 11 to facilitate processing. In other embodiments, housing 10 can also be formed by combining a plurality of plates.

In one embodiment, when the air inlet 112 is arranged at the bottom of the housing 10, the corresponding air inlet 112 is arranged on the bottom housing 11. Similarly, when the air outlet 115 is arranged at the bottom of the housing 10, the air outlet 115 can be arranged on the bottom housing 11. When the air inlet 112 is arranged on the top of the housing 10, the corresponding air inlet 112 is arranged on the casing 12.

In one embodiment, referring to FIG. 1, a control panel 52 is arranged on the front side of the housing 10, and the control panel 52 is inclined from top to bottom toward the rear side of the housing 10 to facilitate user operations and determine the position of the control panel 52. In one embodiment, the control panel 52 is arranged on the casing 12 to facilitate processing.

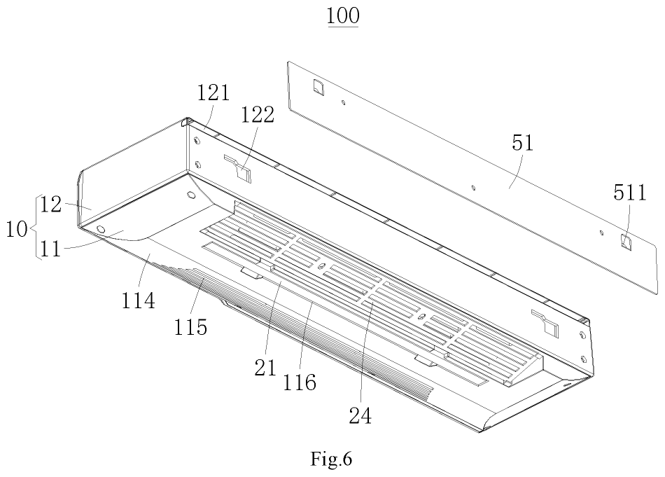

In an embodiment, referring to FIG. 6. FIG. 6 is a schematic diagram of the wall-mounted air purifier 100 when the hanging plate 51 is separated from the housing 10 in this embodiment. A hanging buckle 122 is arranged on the rear side of the housing 10, so that the housing 10 can be installed on the wall by a hooking, which is convenient for installation and fixation. In some other embodiments, the housing 10 can also be installed on the wall using screws.

In one embodiment, referring to FIG. 6, the wall-mounted air purifier 100 further includes a hanging plate 51. The hanging plate 51 is provided with a hook 511 that cooperates with the hanging buckle 122, and the hanging plate 51 is used to be fixed on the wall. To arrange the hanging plate 51, the hanging plate 51 is fixed on the wall first, and then the housing 10 is hanged on the hanging plate 51, which is convenient for positioning and installation. The hanging plate 51 can stick on the wall or fixed on the wall by nails.

In one embodiment, referring to FIG. 6, a hidden trunking 121 is arranged on the rear side of the housing 10 so that the power supply can be arranged in the hidden trunking 121 to facilitate wiring.

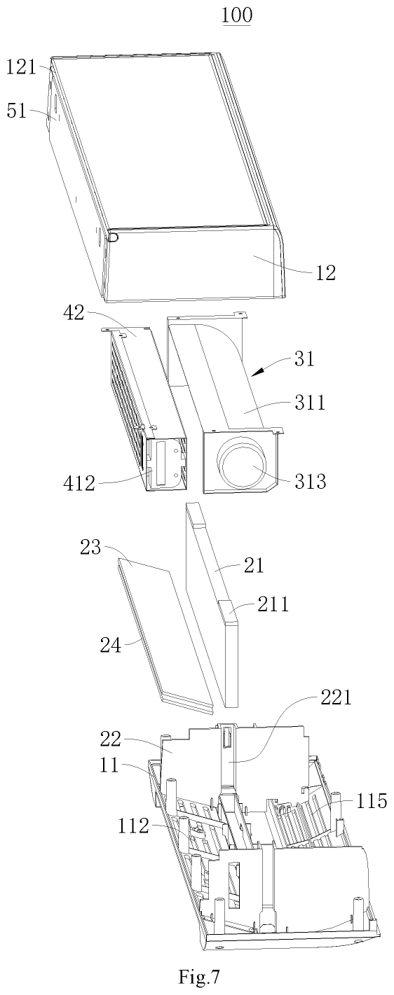

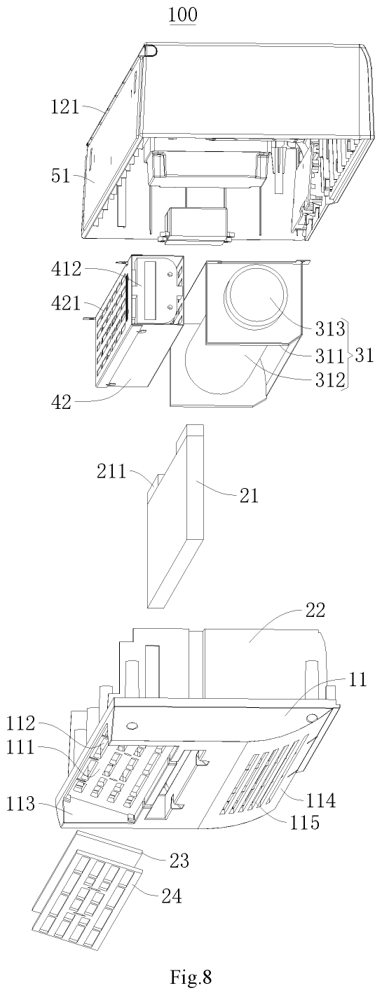

Referring to FIG. 7 and FIG. 8 together. FIG. 7 is an exploded schematic diagram of the wall-mounted air purifier 100 of this embodiment from an angle. FIG. 8 is an exploded schematic diagram of the wall-mounted air purifier 100 of this embodiment from another angle.

In one embodiment, referring to FIGS. 2, 7 and 8, housing 10 is provided with a socket 116, the socket 116 is used for plugging and unplugging the filter 21, the filter 21 is detachably inserted into housing 10 through the socket 116, that is, the filter 21 is inserted into the housing 10 from the socket 116 to facilitate the installation of the filter 21; the filter 21 can also be extracted from the socket 116 to clean or replace the filter 21. In other embodiments, housing 10 can also be disassembled to disassemble filter 21.

In one embodiment, the socket 116 is arranged on the bottom surface of the housing 10. Since the wall-mounted air purifier 100 is fixed on the wall in use, a socket 116 is arranged on the bottom surface of the housing 10 to facilitate the insertion and removal of the filter 21 from the socket 116, and facilitate the disassembly and replacement of the filter 21. In other embodiments, a socket 116 can also be arranged on the side of the housing 10, and the filter 21 can be inserted and removed from the side of the housing 10.

In one embodiment, the filter 21 is vertically arranged in the housing 10 to facilitate the insertion and removal of the filter 21 from the socket 116. In addition, the space occupied by the filter 21 in the front and rear directions of the housing 10 can be reduced, and the width of the housing 10 in the front and rear directions can be reduced.

In one embodiment, a magnetic member 212 is arranged in the housing 10, and a magnetic block 211 is arranged on the filter 21. The magnetic member 212 is connected to the magnetic block 211 by matching with the magnetic attraction, so that the filter 21 is magnetically fixed in the housing 10 to facilitate the disassembly and assembly of the filter 21. In one embodiment, the magnetic member 212 is a magnet, and the magnetic block 211 may be a magnet with magnetic attraction, such as a magnet matched with the magnetic member 212 by magnetic attraction. The magnetic member 212 may also be a soft magnet such as an iron block. The magnetic block 211 is a magnet, and the magnetic member 212 may be a magnet with magnetic attraction, such as a magnet matched with the magnetic block 211. The magnetic block 211 can also be a soft magnet such as an iron block.

In an embodiment, a buckle may be arranged in housing 10 to clamp filter 21 in housing 10, and may also facilitate the disassembly and assembly of filter 21.

In one embodiment, referring to FIGS. 7 and 8, there are two partition plates 22 in housing 10, the two partition plates 22 are arranged at both ends of filter 21, and the partition plate 22 is provided with chute 221, so that when filter 21 is disassembled and assembled, the filter 21 could be guided, and when filter 21 is installed in housing 10, filter 21 can be positioned. In addition, by installing a partition plate 22, all the air entering the housing 10 can pass through the filter 21 to better purify the air. In addition, the partition plate 22 can also increase the strength of the housing 10.

In one embodiment, the air inlet 112 and fan 31 are arranged between the two partition plates 22, so that the air entering from the air inlet 112 will be purified and filtered by the filter 21 to ensure the air purification effect.

In one embodiment, referring to FIGS. 5, 7 and 8, the fan 31 includes wind a cover 311, a through-flow wind wheel 312, and a motor 313. The motor 313 is connected to through-flow wind wheel 312 to drive through-flow wind wheel 312 to rotate through motor 313. The through-flow wind wheel 312 is installed in the wind cover 311, and the airflow is guided by the wind cover 311. Using through-flow wind wheel 312, the volume can be made smaller, and the air volume can be large, and the volume of the housing 10 can be made smaller. In other embodiments, other fan 31 structures can also be used.

In one embodiment, referring to FIG. 4, FIG. 5 and FIG. 7. The housing 10 is also provided with a light shield 42, which covers on the ultraviolet light source 41. By the arrangement of the light shield 42, the ultraviolet light beam emitted by the ultraviolet light source 41 can be better bound in a certain space to increase the intensity of the ultraviolet light in the space, so that the ultraviolet light can sterilize the air in the space and enhance the effect of ultraviolet light sterilization.

In one embodiment, the ultraviolet light source 41 is arranged between the air inlet 112 and the filter 21, so that the air will be irradiated and sterilized by ultraviolet light before entering the filter 21 to better kill the bacteria in the air and avoid the growth of bacteria and other organisms in the filter 21, thus playing a certain role in protecting the filter 21.

In one embodiment, the light shield 42 shields the side of the ultraviolet light source 41 close to the air inlet 112, so as to prevent the ultraviolet light emitted by the ultraviolet light source 41 from leaking from the air inlet 112 and improve safety.

In an embodiment, referring to FIG. 4, FIG. 5 and FIG. 8, the light shield 42 shields the ultraviolet light source 41, and the front and rear sides of the light shield 42 are respectively provided with a vent 421, which can better prevent the ultraviolet light from leaking from the air inlet 112, improve safety and concentrate the ultraviolet light emitted by the ultraviolet light source 41 in the light shield 42 to better sterilize the air passing through the light shield 42.

In one embodiment, the light shield 42 adopts two U-shaped covers in cross section to buckle each other to shield the ultraviolet light source 41, which is convenient for manufacturing. The light shield 42 can also use a cylindrical cover, and the ultraviolet light source 41 is directly supported in the light shield 42.

In one embodiment, the ultraviolet light source 41 includes an ultraviolet tube 411 and a lamp holder 412. The lamp holder 412 is installed in the housing 10, and the lamp holder 412 is protected by the housing 10. The ultraviolet tube 411 is installed on the lamp holder 412, and the ultraviolet tube 411 is supported by the lamp holder 412, and power is supplied to the ultraviolet tube 411 so that the ultraviolet tube 411 emits ultraviolet light. The use ultraviolet tube 411 is of low cost and more uniform light emission.

In one embodiment, the ultraviolet tube 411 can be detachably installed on the lamp holder 412 so as to replace the ultraviolet tube 411 and facilitate maintenance.

In one embodiment, the inner surface of the light shield 42 is provided with a reflective layer, so as to better reflect the ultraviolet light emitted by the ultraviolet light source 41 to the limited space and disinfect and purify the air in the space.

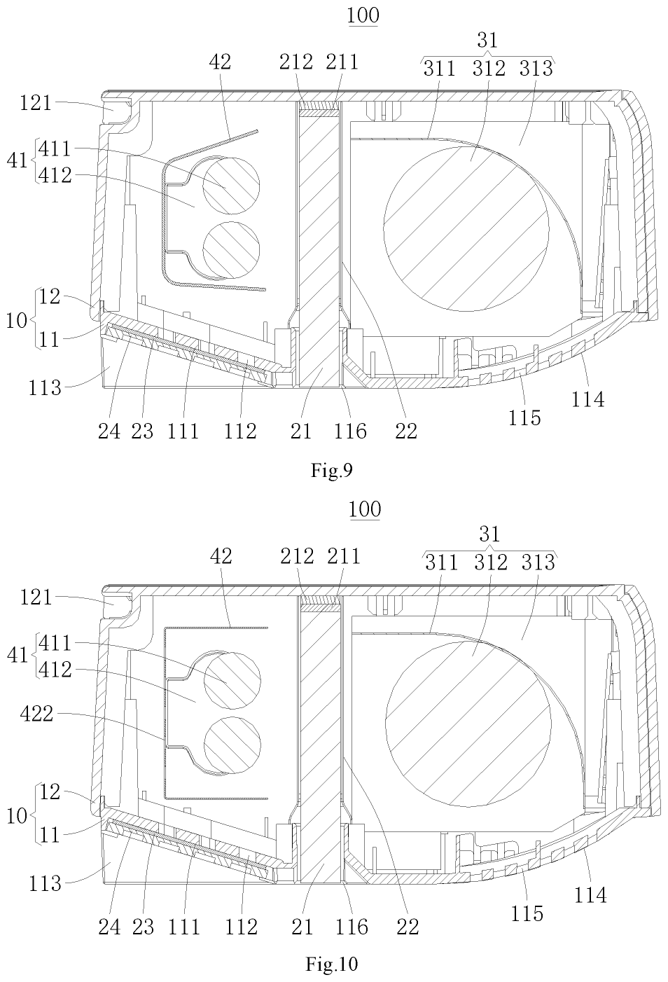

In an embodiment, referring to FIG. 9, which is a schematic sectional view of the wall-mounted air purifier 100 of this embodiment. The side of the light shield 42 close to the filter 21 is open. This structure can not only sterilize the air passing through the filter 21, but also sterilize the filter 21, which can better reduce the growth of bacteria on the filter 21 and improve the air purification effect.

In one embodiment, the light shield 42 can use a U-shaped cover with a simple structure, which is convenient to process and manufacture, low cost, and convenient installation.

In one embodiment, the ultraviolet light source 41 is arranged on the side of the filter 21 close to the air inlet 112, the light shield 42 is open on the side close to the filter 21, and the side of the light shield 42 can also shield the ultraviolet light source 41 close to the air inlet 112 to avoid ultraviolet light leaking from the air inlet 112.

In one embodiment, referring to FIG. 10, which is a schematic sectional view of the wall-mounted air purifier 100 of this embodiment. In this embodiment, the other side of the light shield 42 is provided with a first opening 422, that is, the side of the light shield 42 away from the filter 21 is provided with a first opening 422, so that air can flow to the filter 21 through the first opening 422, reducing air resistance and improving air purification capacity.

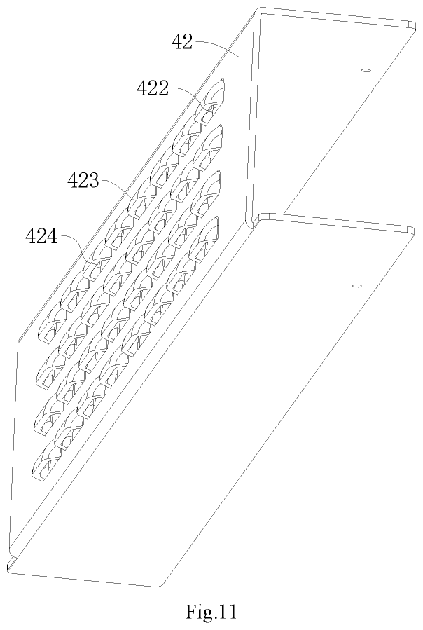

In an embodiment, referring to FIG. 11, which is a schematic diagram of the light shield 42 of this embodiment. The light shield 42 is provided with a blocking sheet 423, the blocking sheet 423 shields the first opening 422, and a second opening 424 communicates with the first opening 422 is formed between the blocking sheet 423 and the light shield 42. The blocking sheet 423 is arranged to block the first opening 422 which can better avoid the leakage of the ultraviolet light source 41 and improve safety.

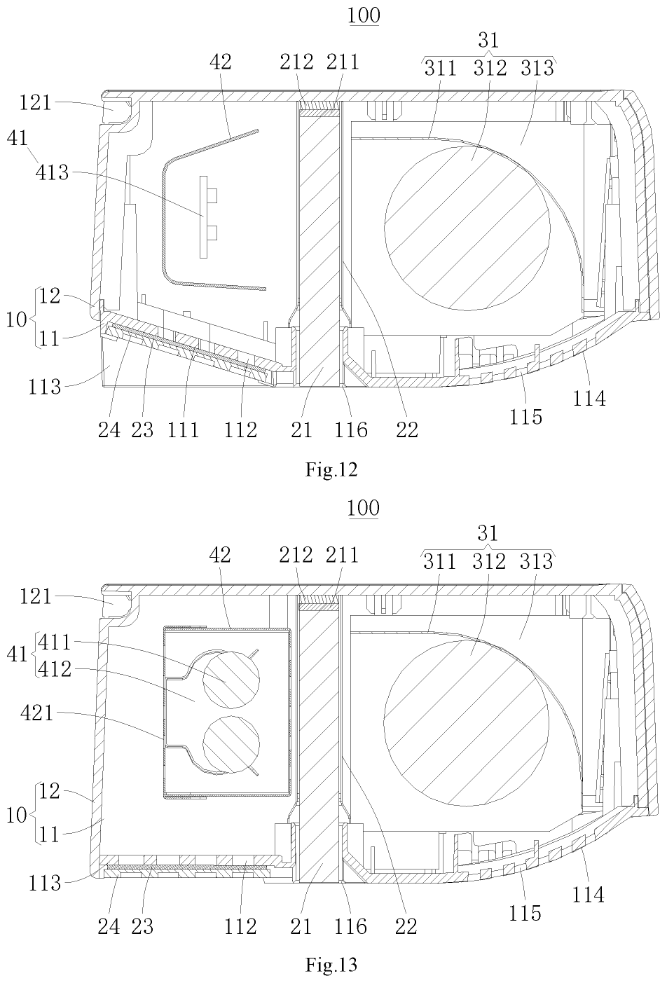

In one embodiment, referring to FIG. 12, which is a schematic sectional view of the wall-mounted air purifier 100 of this embodiment. In this embodiment, the ultraviolet light source 41 includes an ultraviolet LED module 413, and the ultraviolet LED module 413 is installed in the housing 10. The use ultraviolet LED module 413 is more energy-saving and longer life.

In an embodiment, referring to FIG. 13, which is a schematic sectional view of the wall-mounted air purifier 100 of this embodiment. In this embodiment, the rear side of the bottom of housing 10 is flat, and the air inlet 112 is arranged on the rear of the bottom of housing 10. The structure is easy to manufacture.

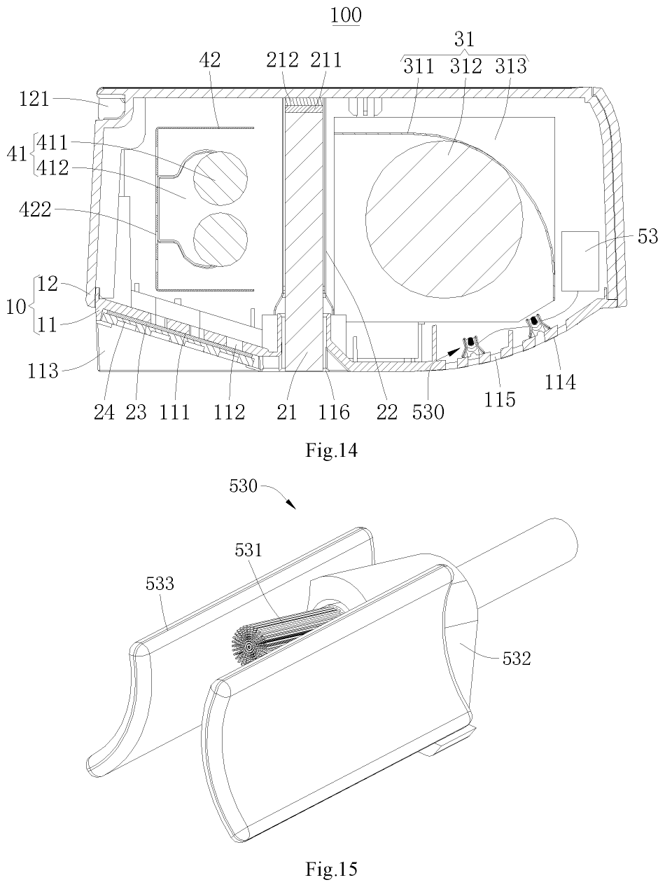

In an embodiment, referring to FIGS. 14 and 15. FIG. 14 is a schematic sectional view of the wall-mounted air purifier 100 of this embodiment. FIG. 15 is a schematic diagram of the transmitting head of this embodiment. In this embodiment, the wall-mounted air purifier 100 also includes a negative ion generator 53, which has a transmitting head 530, and the transmitting head 530 of the negative ion generator 53 is arranged at a position corresponding to the air outlet 115, so that the air flow generated by the fan 31 passes through the transmitting head 530 of the negative ion generator 53 and then flows out of the air outlet 115 to form an airflow with negative ions to better disinfect and purify the air.

In one embodiment, the transmitting head 530 includes a negative ion release brush 531, a support 532, and two guide plates 533. The support 532 is installed on the housing 10, and the negative ion release brush 531 is installed on the support 532. The negative ion release brush 531 is supported by the support 532, and the negative ion release brush 531 extends to the air outlet 115. The two guide plates 533 are respectively arranged opposite to the negative ion release brush 531. On both sides, the distance between the two guide plates 533 gradually expands from the middle of the guide plate 533 downwards, that is, the distance between the two guide plates 533 expands gradually from the middle of the guide plate 533 to the lower side of the guide plate 533. So that the space between the lower parts of the two guide plates 533 gradually increases, and the air flow to the lower part of the two guide plates 533 will slow down and diffuse, so that the negative ions in the air flow can be better diffused to increase the area covered by the negative ions.

In one embodiment, the guide plate 533 and the support 532 are fixedly connected to facilitate the installation and fixation of the guide plate 533, and further facilitate the installation and fixation of the transmitting head 530. In some embodiments, the guide plate 533 can also be separately supported in the housing 10.

In one embodiment, the distance between the two guide plates 533 is gradually expanded upward from the middle of the guide plate 533, that is, the distance between the two guide plates 533 is gradually expanded from the middle of the guide plate 533 to the upper side of the guide plate 533 to allow more air to enter between the two guide plates 533 and to make more air contact the negative ion release brush 531 to generate more negative ions.

In one embodiment, the distance between the upper sides of the two guide plates 533 is smaller than the distance between the lower sides of the two guide plates 533, so as to ensure that the airflow velocity from the lower sides of the two guide plates 533 is less than that of the airflow outside the two outer guide plates 533, which will form a certain siphon effect on the lower side of the two guide plates 533 to better diffuse the negative ions in the air flow between the two guide plates 533, thereby increasing the area covered by the negative ions.

In one embodiment, the negative ion release brush 531 is arranged at a position corresponding to above the middle of the guide plate 533, so that more air flow passes through the negative ion release brush 531, and then is guided and diffused by the guide plate 533.

In one embodiment, the negative ion release brush 531 extends obliquely upward, that is, the negative ion release brush 531 extends obliquely upward from the support 532 to the free end of the negative ion release brush 531. Since the free end of the negative ion release brush 531 is relatively more open, the extending the negative ion release brush 531 obliquely upwards can reduce the resistance of the negative ion release brush 531 to the air flow, thereby facilitating the air flow through the negative ion release brush 531 to produce more negative ions.

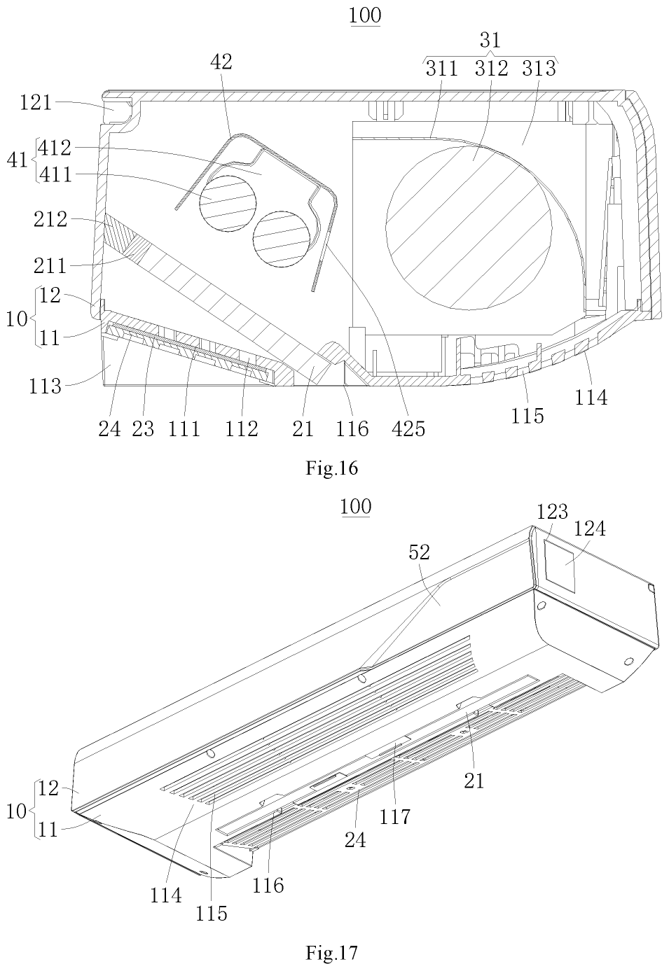

In an embodiment, referring to FIG. 16, which is a schematic sectional view of the wall-mounted air purifier 100 of this embodiment, the ultraviolet light source 41 is arranged between the fan 31 and the filter 21, and the air filtered and purified by the filter 21 is then irradiated and disinfected by the ultraviolet light source 41 to ensure the purification effect of the air. In addition, the ultraviolet light source 41 is arranged between the fan 31 and the filter 21, and the filter 21 can also be used to block the ultraviolet light to prevent the ultraviolet light from leaking from the air inlet 112.

In one embodiment, referring to FIG. 16, the light shield 42 is provided with a third opening 425 so that the air entering the light shield 42 after being filtered by the filter 21 can better flow to the fan 31 and reduce the air resistance.

In one embodiment, referring to FIG. 17, which is a perspective schematic diagram of the wall-mounted air purifier 100 of this embodiment, the socket 116 is arranged at the bottom of the housing 10, the socket 116 is provided with a sliding plate 117, and the sliding plate 117 is slidably installed at the bottom of the housing 10. When the filter 21 is inserted into the housing 10 through the socket 116, the sliding plate 117 can be pulled out so that the sliding plate 117 supports the filter 21; When disassembling the filter 21, sliding the sliding plate 117 so that the sliding plate 117 is received in the housing 10, in order to remove the filter 21.

In one embodiment, referring to FIG. 5 and FIG. 17, the ultraviolet tube 411 is detachably installed on the lamp holder 412, and the side of the housing 10 is provided with an opening window 123, and the housing 10 is provided with a cover plate 124 that covers the opening window 123. Arranging the opening window 123 on the housing 10 so that the ultraviolet tube 411 can be plugged and unplugged from the opening window 123 to facilitate the replacement of the ultraviolet tube 411.

The wall-mounted air purifier 100 of the embodiment of the present application can not only filter purify, but also sterilize purify, and has a large air intake, which can provide purified air to the user's surroundings faster.

The above are only optional embodiments of the present application and are not used to limit the present application. Any modification, equivalent replacement and improvement made within the spirit and principles of the present application shall be within the protection scope of the present application.

What is claimed is:

1. A wall-mounted air purifier, comprising a housing, a filter installed in the housing, and a fan installed in the housing; wherein the housing is provided with an air inlet and an air outlet, the air outlet is arranged at a position corresponding to an outlet of the fan, the filter is arranged between the air inlet and the fan, and the air inlet is arranged at a position corresponding to a rear side of a bottom of the housing, and the air outlet is arranged at a position corresponding to a front side of the bottom of the housing.

2. The wall-mounted air purifier of claim 1, wherein the bottom of the housing has an inclined surface extending backward and upward, and the air inlet is arranged on the inclined surface.

3. The wall-mounted air purifier of claim 2, wherein the bottom of the housing is provided with baffles protruding downward at both ends of the inclined surface respectively.

4. The wall-mounted air purifier of claim 1, wherein the wall-mounted air purifier further comprises a filter screen covering the air inlet, and the filter screen is detachably installed on the housing.

5. The wall-mounted air purifier of claim 2, wherein the wall-mounted air purifier further comprises a filter screen covering the air inlet, and the filter screen is detachably installed on the housing.

6. The wall-mounted air purifier of claim 3, wherein the wall-mounted air purifier further comprises a filter screen covering the air inlet, and the filter screen is detachably installed on the housing.

7. The wall-mounted air purifier of claim 4, wherein the wall-mounted air purifier further comprises a grid plate for supporting the filter screen, and the grid plate is detachably installed on the housing.

8. The wall-mounted air purifier of claim 5, wherein the wall-mounted air purifier further comprises a grid plate for supporting the filter screen, and the grid plate is detachably installed on the housing.

9. The wall-mounted air purifier of claim 6, wherein the wall-mounted air purifier further comprises a grid plate for supporting the filter screen, and the grid plate is detachably installed on the housing.

10. The wall-mounted air purifier of claim 1, wherein the bottom of the housing has an air outlet surface extending forward and upward, and the air outlet is defined in air outlet surface.

11. The wall-mounted air purifier of claim 2, wherein the bottom of the housing has an air outlet surface extending forward and upward, and the air outlet is defined in the air outlet surface.

12. The wall-mounted air purifier of claim 3, wherein the bottom of the housing has an air outlet surface extending forward and upward, and the air outlet is defined in the air outlet surface.

13. The wall-mounted air purifier of claim 10, wherein the air outlet surface is arcuate, or the air outlet surface is inclined.

14. The wall-mounted air purifier of claim 11, wherein the air outlet surface is arcuate, or the air outlet surface is inclined.

15. The wall-mounted air purifier of claim 12, wherein the air outlet surface is arcuate, or the air outlet surface is inclined.

16. The wall-mounted air purifier of claim 1, wherein an ultraviolet light source is further installed in the housing, and the ultraviolet light source is arranged between the air inlet and the fan.

17. The wall-mounted air purifier of claim 2, wherein an ultraviolet light source is further installed in the housing, and the ultraviolet light source is arranged between the air inlet and the fan.

18. The wall-mounted air purifier of claim 3, wherein an ultraviolet light source is further installed in the housing, and the ultraviolet light source is arranged between the air inlet and the fan.

19. The wall-mounted air purifier of claim 1, wherein a control panel is arranged on the front side of the housing, and the control panel is inclined from top to bottom toward the rear side of the housing.

20. The wall-mounted air purifier of claim 1, wherein the fan comprises a wind cover installed in the housing, a through-flow wind wheel installed in the wind cover, and a motor that drives the wind wheel to rotate.
